(19) United States
(12) Patent Application Publication
(10) Pub. No.: US 2024/0232576 A1
(43) Pub. Date: Jul. 11, 2024

(54) METHODS AND SYSTEMS FOR DETERMINING PHYSICAL PROPERTIES VIA MACHINE LEARNING

(71) Applicant: THE REGENTS OF THE UNIVERSITY OF CALIFORNIA, Oakland, CA (US)

(72) Inventors: Teresa Head-Gordon, Oakland, CA (US); Mojtaba Haghighatlari, Cambridge, MA (US)

(21) Appl. No.: 18/681,470

(22) PCT Filed: Aug. 4, 2022
(86) PCT No.: PCT/US2022/074526

§ 371 Date: Feb. 5, 2024

Related U.S. Application Data
(60) Provisional application No. 63/203,937, filed on Aug. 4, 2021.
(60) Provisional application No. 63/260,263, filed on Aug. 13, 2021.

(51) Int. Cl. G06N 3/04; G06N 3/084; G16C 60/00

(52) U.S. Cl. CPC G06N 3/04; G06N 3/084; G16C 60/00

(57) ABSTRACT

Systems and methods are provided for a message passing neural network (MPNN). In one example, the MPNN is a geometric MPNN which utilizes Newton's equations of motion to learn interatomic potentials and forces. Specifically, by leveraging directional information from trainable latent force vectors and physics-infused operators based on Newtonian physics, the geometric MPNN may remain rotationally equivariant and many-body interactions may be inferred by readily interpretable physical features with increased data efficiency relative to other deep learning models. Such many-body interactions may include reactive and non-reactive ab initio datasets (e.g., single small molecule dynamics, a large set of chemically diverse molecules, and methane and hydrogen combustion reactions). In this way, higher performance results on physical properties such as energies and forces may be achieved with greater data efficiency and computational efficiency relative to other deep learning models.

### CROSS REFERENCE TO RELATED APPLICATIONS

The present application claims priority to U.S. provisional Application No. 63/203,937, entitled “MACHINE LEARNING OF PHYSICAL PROPERTIES USING AB INITIO CHEMISTRY”, filed on Aug. 4, 2021. The present application also claims priority to U.S. provisional Application No. 63/260,263, entitled “METHODS AND SYSTEMS FOR DETERMINING PHYSICAL PROPERTIES VIA MACHINE LEARNING”, filed on Aug. 13, 2021. The entire contents of the above-listed applications are hereby incorporated by reference for all purposes.

### TECHNICAL FIELD

The present description relates generally to systems and methods for determining physical properties of molecules and chemical reactions via machine learning models, particularly via geometric neural networks.

### BACKGROUND AND SUMMARY

The application of machine learning (ML) models to predict ab initio potential energies and forces for bulk silicon has inspired the modern cheminformatics era (Behler and Parrinello, Phys. Rev. Lett., 98:146401, 2007). Specifically, approximating the total potential energy as a sum of atomic energies paved the way for hierarchical decomposition of structural and physical features of molecules in order to provide atom-centric feature representations, which may address different permutations and/or numbers of atoms in molecules with relative ease. With such strategies, ML modeling may be capable of good to accurate predictions of molecular energy and atomic forces. However, ML models have typically required prodigious quantities of data to achieve sufficient accuracy, as exemplified by the 57000 small CHNO-containing molecules perturbed into 22 million different configurations and energies evaluated with density functional theory (DFT) data from the “ANI” datasets.

In some earlier artificial neural networks (ANNs) and kernel methods used to predict energies and (vectorial) forces, atomic environments may be modeled using symmetry functions that rely on two-body and higher-order correlated features, e.g., distances, angles, dihedrals, etc., for any central atom. Such “handcrafted” many-body representations have shown improvements in predicting interatomic potentials and directional forces, but may be subject to rising computational costs of incorporating three-body representations and higher.

Alternatively, message passing neural networks (MPNN) replace the handcrafted features (e.g., the distances and angles) with trainable operators that may rely on atomic numbers and positions alone, such that a resultant learned latent space of such MPNNs has an added advantage in chemical accuracy as compared to explicit symmetry functions. One MPNN method for three-dimensional (3D) structures is SchNet, which takes advantage of a continuous filter layer that may facilitate convolution of decomposed interatomic distances with atomic attributes. Related methods have subsequently built on such successes to incorporate additional features to describe atomic environments. For example, PhysNet adds prior knowledge about long-range electrostatics in energy predictions and DimeNet takes advantage of angular information and higher stability basis functions based on Bessel functions. In standard MPNNs, however, representations may be reduced to transformationally identical features, for example, quantities that are invariant to translation and permutation. However, by limiting such representations of atomic features to be invariant to transformations of the system, all such models, including MPNNs, may fail to distinguish systems even if represented by up to four-body information.

Because the disclosed technology predicts both energies and force vectors, and given that vectorial features may be affected by transformation T of input structure x∈X, an output of each operator may desirably reflect such transformation equivalently when needed. More specifically, rotational transformations (such as through angular displacements ϕ) are an outstanding challenge in modeling of 3D objects, and learning a global orientation of structures for higher-level trajectories [e.g., molecular dynamics (MD) trajectories] with many molecules has proven difficult or infeasible. That is, the challenge is to ensure that a function ϕ: X→Y is equivariant to a transformation group g, which is satisfied if:

$$ϕ ( {T}_{g} ( x ) ) = {T}_{g}^{′} ( ϕ ( x ) ) \qquad ( 1 )$$

where T_{g} and T_{g}′ are equivalent transformations in the (abstract) group g acting on an input space and an output space, respectively.

At least some neural networks that are equivariant to transformations in Euclidean space have been found to improve ML predictive performance when evaluated on a variety of tasks while reducing on excessively large quantities of ab initio reference data. For instance, ML models have introduced multipole expansions (e.g., NequIP) or have been designed to take advantage of precomputed features and/or higher-order tensors using molecular orbitals. In spite of infusing additional physical knowledge into ML models, a computational cost of spherical harmonics and availability/versatility of precomputed features, or a lack of physical interpretability, may prove limiting (PaiNN is one such MPNN model which satisfies equivariance, but in which mathematical operations do not follow a physically interpretable procedure). An equivariant model would desirably be equipped with appropriate strategies to decrease a rank of equivariant features employed throughout an architecture of the model to be computationally viable while also retaining test set accuracy.

Herein, a geometric MPNN based on Newton's equations of motion that also achieves equivariance with respect to physically relevant permutations is provided. Specifically, the geometric MPNN may improve a capacity of structural information therein by generating latent force vectors based on Newton's third law. A force direction may help to describe an influence of neighboring atoms on a central atom based on directional positions of the atoms in 3D coordinate space with respect to one other. In this way, by introducing vector features as attributes of the atoms, an underlying representation may remain equivariant to rotations in the 3D coordinate space and preserve the vector features throughout the geometric MPNN. Further, by infusing more physical properties into an architecture of the geometric MPNN, the geometric MPNN may enable modeling of reactive and non-reactive chemistry with competitive or superior computational performance to prior equivariant (e.g., invariant-only) models with significantly less demand on training dataset size (e.g., 1-10% of prior training dataset sizes utilized to achieve comparable accuracy).

In one example, a method for a trained neural network may include acquiring a plurality of inputs including a plurality of initial parameters for a polyatomic system, passing the plurality of inputs through one or more rotationally equivariant message passing layers of the trained neural network to generate a plurality of outputs including an update to each of the plurality of inputs, and determining a potential energy of the polyatomic system based on the plurality of outputs, wherein each of the one or more rotationally equivariant message passing layers may be constructed from one or more symmetric message functions.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1A shows a schematic diagram of Newton's laws of motion applied to an atomic system, including force and displacement vectors for a central atom with respect to a plurality of neighboring atoms, according to an embodiment of the present disclosure.

FIG. 1B shows a schematic diagram of a message passing layer included in a geometric message passing neural network (MPNN), according to an embodiment of the present disclosure.

FIG. 1C shows a schematic diagram of a computing device, the computing device storing the geometric MPNN including one or more message passing layers, according to an embodiment of the present disclosure.

FIG. 2 shows a plot of mean absolute error in energy and forces as a function of a number of independent hydrogen combustion reaction samples determined via two exemplary neural networks.

FIG. 3 shows a schematic diagram of latent force vectors and normalized reference vectors exerted on each atom of an aspirin molecule at three message passing layers of an exemplary geometric MPNN.

### DETAILED DESCRIPTION

The following description relates to systems and methods for determining physical properties of atomic systems (e.g., polyatomic chemical and biochemical systems) via a machine learning (ML) model. In an exemplary embodiment, the ML model may include a geometric neural network, such as a geometric message passing neural network (MPNN). In geometric neural networks, data may be represented as interconnected nodes of a graph. Accordingly, and in contrast to other (e.g., non-geometric) ML models, such data may not be assumed independent.

Graph representations may be useful for physical systems described by many-body interactions. For example, to obtain desirably accurate ab initio representations of polyatomic systems, physical properties thereof may depend upon positions of two or more atoms within the system [rigorously, a given physical property may be influenced by each atom in the system (“N-body interactions,” where N is a number of bodies in the system), but in practice assumptions may be made]. Accordingly, a graph representation of a polyatomic system may be formulated (referred to herein as a “molecular graph”), upon which a message passing layer of a geometric MPNN may be constructed.

In an exemplary embodiment, given a molecular graph G with (“one-body”) atomic features aᵢ∈ [symbol] ^{nf} (where nf is a number of features) and interatomic attributes eᵢⱼ∈ [symbol] ^{b} (where b is a number of basis functions), a message passing layer may be defined as:

$${m}_{ij} = {M}_{l} ( {a}_{i}^{l} , {a}_{j}^{l} , {e}_{ij} ) \qquad ( 2 )$$

$${m}_{i} = {Σ}_{j ∈ 𝒩 ( i )}^{j} {m}_{ij} \qquad ( 3 )$$

$${a}_{i}^{l + 1} = {U}_{l} ( {a}_{i}^{l} , {m}_{i} ) \qquad ( 4 )$$

where mᵢⱼ is referred to as “the message,” Mₗ is referred to as “the message function,” subscripts and superscripts/accounts for a number of times the message passing layer operates iteratively, Uₗ is referred to as “the update function,” and [symbol] (i) is all neighboring atoms for atom i (such that mᵢ is the sum of messages mᵢⱼ over all neighboring atoms for the atom i). In one example, a combination of explicit differentiable functions and operators with trainable parameters may be utilized for Mₗ and Uₗ.

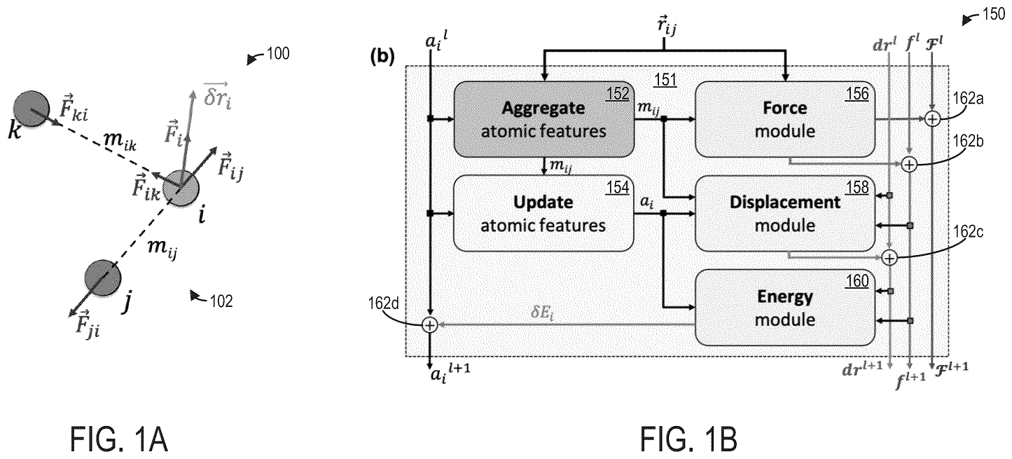

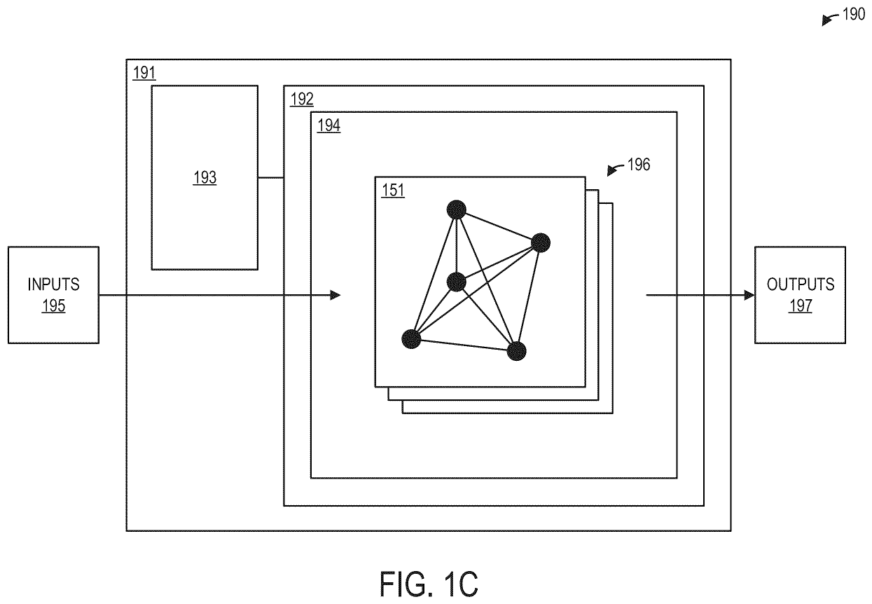

The geometric MPNN may consider a molecular graph (e.g., the molecular graph g) defined by atomic numbers Zᵢ∈ [symbol] ¹ and relative position vectors \vec{rᵢⱼ}=\vec{rⱼ}−\vec{rᵢ}∈ [symbol] ³ as input and applying operations inspired by Newton's equations of motion (also referred to herein as “Newton's laws of motion”) to create feature arrays aᵢ∈ [symbol] ^{nf} to represent each atom in its immediate environment, while remaining invariant to rotations of the input configuration. Referring now to FIG. 1A, a schematic diagram 100 depicting Newton's laws of motion applied to an atomic system 102, including force vectors (e.g., force vectors \vec{Fᵢ}, \vec{Fᵢⱼ}, \vec{Fⱼᵢ}, \vec{Fᵢₖ}, and \vec{Fₖᵢ}) and displacement vectors (e.g., displacement vector \vec{δrᵢ}) for a central atom i with respect to a plurality of neighboring atoms (e.g., atoms j and k), is shown. Specifically, the force vector Fᵢ may represent a total force on the atom i and the force vector \vec{Fij} may represent a force on the atom i from the neighboring atom j. Messages mᵢⱼ and mᵢₖ are further shown, representing messages between the atoms i and j and the atoms i and k, respectively.

To determine the feature arrays (e.g., aᵢ), the geometric MPNN may take advantage of multiple layers of message passing which are rotationally equivariant, wherein each layer may consist of multiple modules that include operators to construct force and displacement feature vectors, which may be contracted to the feature arrays via an energy calculator module. Referring now to FIG. 1B, a schematic diagram 150 depicting a message passing layer 151 included in a geometric MPNN is shown. The message passing layer 151 may be one of one or more message passing layers (as described in detail below with reference to FIG. 1C), each given message passing layer 151 updating atomic feature arrays aᵢ^{l} for a central atom i, latent force vectors [symbol] , force vectors (f^{l} and F^{l}), and displacement vectors (dr^{l}) (where l indexes the given message passing layer 151). Specifically, within the given message passing layer 151, a plurality of modules may perform the updating (e.g., from l to l+1) based on the atomic feature arrays aᵢ^{l}, the latent force vectors [symbol] , the force vectors f^{l} and F^{l}, the displacement vectors dr^{l}, and interatomic distances \vec{rᵢⱼ} between the central atom i and each neighboring atom j. As shown, and as discussed in greater detail below, the plurality of modules may at least include an atomic feature aggregator module 152, an atomic feature updater module 154, a force module 156, a momentum module 158, and an energy module 160. Concatenations are indicated at 162a, 162b, 162c, and 162d.

The geometric MPNN of the present disclosure leverages projection of equivariant feature vectors to invariant arrays to predict potential energies (which are invariant to rotations of atomic configurations). Consequently, the geometric MPNN may avoid any many-to-one mapping of rotationally equivariant features to invariant energies. In this way, the iterative message passing of atomic environments may update the feature array aᵢ^{l} that represents each central atom i in its immediate environment. Such feature representation may remain equivariant to rotations of an initial configuration in the geometric MPNN through the atomic feature aggregator, energy, force and displacement modules 152, 156, 158, and 160. Proof of equivariance of the geometric MPNN of the present disclosure is discussed in greater detail below.

The atomic feature aggregator module 152 may follow a standard message passing and is invariant to rotation. One notable difference to some other MPNNs, however, is use of a symmetric message function between atom pairs (such that mᵢⱼ=mⱼᵢ). In one example, the symmetric message function may incorporate a radial Bessel function, a polynomial cutoff function, and a pair of multilayer perceptrons. The message mᵢⱼ may be used in each of the equivariant modules discussed herein to account for interatomic interactions. For example, atomic features may be initialized based on trainable embedding g: [symbol] ¹→ [symbol] ^{nf} of atomic numbers Zᵢ (aᵢ⁰=g(Zᵢ). Further, an edge function e: [symbol] ³→ [symbol] ^{nb} may be employed to represent the interatomic distances \vec{rᵢⱼ} using radial Bessel functions:

$$e ( \vec{{r}_{ij}} ) = \sqrt{\frac{2}{{r}_{c}}} \frac{sin ( \frac{n π}{{r}_{c}} ‖ \vec{{r}_{ij}} ‖ )}{‖ \vec{{r}_{ij}} ‖} \qquad ( 5 )$$

where re is a cutoff radius and ∥\vec{rᵢⱼ}∥ returns the interatomic distance between any atoms i and j. A self-interaction linear layer ϕ_{rbf}: [symbol] ^{nb}→ [symbol] ^{nf} may be used to combine an output of the radial Bessel functions with one another. Thereafter, an envelope function may be employed to implement a continuous radial cutoff around each atom through use of, in one example, a polynomial function e_{cut} introduced by Klicpera et al. (Klicpera, J.; Gross, J.; and Gunnemann, S., arXiv:2003.03123v1, pp. 1-13, 2020) with a polynomial degree p=7 may be selected. Thus, an edge operation ϕₑ: [symbol] ³→ [symbol] ^{nf} may be defined as a trainable transformation of relative atom position vectors in the cutoff radius r_{c}:

$${ϕ}_{e} ( \vec{{r}_{ij}} ) = {ϕ}_{rbf} ( e ( \vec{{r}_{ij}} ) ) {e}_{cut} ( {r}_{c} , ‖ \vec{{r}_{ij}} ‖ ) \qquad ( 6 )$$

The output of ϕₑ may be rotationally invariant, as it may only depend upon the interatomic distances (e.g., \vec{rᵢⱼ}).

A symmetric message mᵢⱼ may be passed between any pair of atoms. That is, the message that is passed between atom i and atom j may be the same in both directions (mᵢⱼ=mⱼᵢ). The symmetric message mᵢⱼ may be introduced by element-wise products between all feature arrays involved in any two-body interaction:

$${m}_{ij} = {ϕ}_{a} ( {a}_{i}^{l} ) {ϕ}_{a} ( {a}_{j}^{l} ) {ϕ}_{e} ( \vec{{r}_{ij}} ) \qquad ( 7 )$$

where ϕₐ: R^{nf}→R^{nf} indicates a trainable and differentiable multilayer perceptron. It will be appreciated that ϕₐ may be the same function applied to all atoms. Thus, due to weight sharing and multiplication of output features of both atoms of the two-body interaction, the mᵢⱼ may remain symmetric at each layer of message passing. To complete feature array aggregation, equation (3) may be employed to sum all messages mᵢⱼ received by central atom i from its neighbors [symbol] (i). The atomic features at each layer may then be updated (e.g., at the atomic feature updater module 154) using the sum of received messages:

$${a}_{i}^{l + 1} = {a}_{i}^{l} + {Σ}_{j ∈ 𝒩 ( i )}^{j} {m}_{ij} \qquad ( 8 )$$

Directional information may be leveraged at the force module 156, in which a magnitude of the symmetric force may be estimated as a function of mᵢⱼ (∥\vec{Fᵢⱼ}∥=ϕ_{F}(mᵢⱼ)). A product of the force magnitude by unit distance vectors \hat{r}ᵢⱼ=\vec{rᵢⱼ}/∥\vec{rᵢⱼ}∥ null results in antisymmetric interatomic forces that accordingly obey Newton's third law (noting \vec{rᵢⱼ}=−\vec{rᵢⱼ}):

$$\vec{{F}_{{ι}_{J}}^{l}} = {ϕ}_{F} ( {m}_{ij} ) {\hat{r}}_{ij} \qquad ( 9 )$$

where ϕ_{F} is a differentiable learned function. A total force \vec{Fᵢ^{l}} at each message passing layer l on atom i may be determined by summing all forces from the neighboring atoms j in the atomic environment (e.g., F

$$\vec{{F}_{ι}^{l}} = {\sum }_{j ∈ 𝒩 ( i )}^{j} \vec{{F}_{{ι}_{J}}^{l}} \qquad ( 10 )$$

and updating latent force vectors at each layer L (Fᵢ^{l}):

$${ℱ}_{i}^{l + 1} = {ℱ}_{i}^{l} + \vec{{F}_{ι}^{l}} \qquad ( 11 )$$

A latent force vector from a final layer may be used in a loss function to ensure that a corresponding latent space mimics underlying physical rules.

A continuous filter may further be leveraged to decompose and scale the latent force vectors along each dimension using another learned function of. In this way, the vector field may be featurized to avoid excess abstraction in the structural information carried therein:

$$Δ {f}_{i} = {\sum }_{j ∈ 𝒩 ( i )}^{j} {ϕ}_{f} ( {m}_{ij} ) \vec{{F}_{{ι}_{J}}^{l}} \qquad ( 12 )$$

As a result, the constructed latent interatomic forces may be decomposed by rotationally invariant features along each dimension, which are referred to herein as “feature vectors” (fᵢ^{l}). Following message passing, the force feature vectors may be updated with Δfᵢ after each layer:

$${f}_{i}^{l + 1} = {f}_{i}^{l} + Δ {f}_{i} \qquad ( 13 )$$

Inspired by Newton's second law (e.g., in that forces update displacements), the displacement module 158 may approximate a displacement factor using a learned function ϕᵣ that acts on a current state of each atom presented by atomic features thereof (aᵢ^{l+1}):

$$δ {r}_{i} = {ϕ}_{r} ( {a}_{i}^{l + 1} ) {f}_{i}^{l + 1} \qquad ( 14 )$$

Displacement feature vectors (e.g., drᵢ^{l}) may be updated by δrᵢ and a weighted sum of all atomic displacements from the previous layer. The weights may be estimated based on a trainable function of messages (ϕᵣ′) between the atoms:

$${dr}_{i}^{l + 1} = {\sum }_{j ∈ 𝒩 ( i )}^{j} {ϕ}_{r}^{′} ( {m}_{ij} ) {dr}_{j}^{l} + δ {r}_{i} \qquad ( 15 )$$

The weight component functions similarly to an attention mechanism to concentrate on two-body interactions that cause maximum movement in the atoms. Since (initial) forces at l=0 are zero (fᵢ⁰=0), the displacements may also be initialized with zero values (drᵢ⁰=0).

The energy module 160 may contract the directional information to rotationally invariant atomic features. By basing the force and displacement modules 156, 158 on Newton's equations of motion, the potential energy change OU; may be approximated for each atom using fᵢ^{l} and δrᵢ^{l} (considering that fᵢ^{l}≈−δU/δrᵢ^{l}). Thus, an energy change for each atom may be determined by:

$$δ {U}_{i} = - {ϕ}_{u} ( {a}_{i}^{l + 1} ) ( {f}_{i}^{l + 1} \cdot {dr}_{i}^{l + 1} ) \qquad ( 16 )$$

where ϕᵤ is another differentiable learned function that operates on the atomic features and predict an energy coefficient for each atom. A dot product of two feature vectors may contract the features along each dimension to a single feature array. The atomic features may then be further updated using the contracted directional information presented as an atomic potential energy change:

$${a}_{i}^{l + 1} = {a}_{i}^{l + 1} + δ {U}_{i} \qquad ( 17 )$$

In this way, the above provided approach may be both physically and mathematically consistent with rotational equivariance operations: physically, the energy change may constitute a meaningful addition to the atomic feature arrays which may ultimately be used to predict atomic energies; and mathematically, the dot product of two feature vectors contracts the rotationally equivariant features to invariant features similar to Euclidean distances used in the atomic feature aggregator module 152.

The above provided approach may be proved to be rigorously rotationally equivariant on atomic positions Rᵢ∈ [symbol] ³ and atomic numbers Zi for a rotation matrix T∈ [symbol] ^{3×3}. Specifically, in equation (5), the Euclidean distance is invariant to the rotation, as it may be shown that:

$${‖ {Tr}_{ij} ‖}^{2} = {‖ {TR}_{j} - {TR}_{i} ‖}^{2} = {( {R}_{j} - {R}_{i} )}^{T} {T}^{T} T ( {R}_{j} - {R}_{i} ) = {( {R}_{j} - {R}_{i} )}^{T} I ( {R}_{j} - {R}_{i} ) = {‖ {R}_{j} - {R}_{i} ‖}^{2} = {‖ {r}_{ij} ‖}^{2} \qquad ( 18 )$$

(which means that the Euclidean distance is indifferent to rotation of the positions). Consequently, feature arrays mᵢⱼ, aᵢ, and all linear or non-linear functions acting thereon result in invariant outputs. The only assumptions for the proof is that a linear combination of vectors or their product with invariant features will remain rotationally equivariant. Based on this assumption, equations (9) to (15) will remain equivariant to the rotations. For instance, the same rotation matrix T may propagate through equation (9) such that:

$${ϕ}_{F} ( {Tm}_{ij} ) T {\hat{r}}_{ij} = {ϕ}_{F} ( {m}_{ij} ) T {\hat{r}}_{ij} = T {ϕ}_{F} ( {m}_{ij} ) {\hat{r}}_{ij} = T \vec{{F}_{ι}^{l}} \qquad ( 19 )$$

Equation (16) may further remain invariant to the rotations due to the dot product operation. The proof for invariant atomic energy changes is as follows:

$$- {ϕ}_{u} ( {a}_{i}^{l + 1} ) ( {Tf}_{i}^{l + 1} \cdot {Tdr}_{i}^{l + 1} ) = - {ϕ}_{u} ( {a}_{i}^{l + 1} ) ( {f}_{i}^{l + 1} {T}^{T} {Tdr}_{i}^{l + 1} ) = - {ϕ}_{u} ( {a}_{i}^{l + 1} ) ( {f}_{i}^{l + 1} {Idr}_{i}^{l + 1} ) = - {ϕ}_{u} ( {a}_{i}^{l + 1} ) ( {f}_{i}^{l + 1} \cdot {dr}_{i}^{l + 1} ) = δ {U}_{i} \qquad ( 20 )$$

Accordingly, equivariant features may be contracted to invariant arrays. Adding such arrays to the atomic features may preserve the invariance for a final prediction of a total potential energy based on atomic contributions.

Referring now to FIG. 1C, a schematic diagram 190 depicting a computing device 191 storing a geometric MPNN 194 including one or more message passing layers 196 is shown, the one or more message passing layers 196 including the message passing layer 151 of FIG. 1B. Specifically, the computing device 191 may include a storage device or memory 192 storing the geometric MPNN 194 and a processor 193 configured with executable instructions in non-transitory memory for training and executing the various functionalities of the geometric MPNN 194, the processor 193 being communicably coupled to the storage device 192. The storage device 192 may include any known data storage medium, for example, a permanent storage medium, removable storage medium, and the like. Alternatively, the storage device 192 may be a non-transitory storage medium.

The geometric MPNN 194 may receive a plurality of inputs 195 (e.g., initial atomic feature arrays, initial latent force vectors, initial force vectors, initial displacement vectors, and initial interatomic distances), which may be passed through the one or more message passing layers 196 in sequence to generate a plurality of outputs 197 (e.g., final atomic feature arrays, final latent force vectors, final force vectors, final displacement vectors, and final interatomic distances) from which further physical properties may be determined. Since the geometric MPNN 194 is a geometric neural network, message passing in the message passing layer 151 is illustrated in the schematic diagram 190 as a graph.

Further, the final atomic feature arrays (e.g., in the plurality of outputs 198) may be mapped to atomic potential energies. Specifically, and following the summation rule described by Behler and Parrinello, a differentiable function may be used to map the updated atomic features after last layer aᵢ^{L} to atomic potential energies Eᵢ. Ultimately, a total potential energy \tilde{E} may be predicted as a sum of all atomic potential energies Eᵢ:

$${E}_{i} = {ϕ}_{out} ( {a}_{i}^{L} ) \qquad ( 21 )$$

$$\tilde{E} = {\sum }_{i}^{{N}_{m}} {E}_{i} \qquad ( 22 )$$

where Nₘ is the total number of atoms and ϕₒᵤₜ: [symbol] ^{nf}→ [symbol] ¹ is a fully connected network with sigmoid linear unit (SiLU) activation after each layer (excepting the last layer).

Forces may be obtained via a gradient of potential energy with respect to atomic positions. In this way, energy conservation may be guaranteed and atomic forces may be provided for robust training of the atomic environments:

$${\tilde{F}}_{i} = - {∇}_{i} \tilde{E} \qquad ( 23 )$$

The geometric MPNN 194 may be trained using relatively small batches of data with batch size M. A loss function [symbol] may penalize the geometric MPNN 194 for predicted energy values, force components, and a direction of latent force vectors from the last message passing layer (Fᵢ^{L}). The three terms of the loss function [symbol] are formulated as:

$$ ℒ = \frac{{λ}_{E}}{M} {\sum }_{m}^{M} {( {\tilde{E}}_{m} - {E}_{m} )}^{2} + \frac{{λ}_{F}}{M} {\sum }_{m}^{M} \frac{1}{3 {N}_{m}} {\sum }_{i}^{{N}_{m}} {‖ {\tilde{F}}_{mi} - {F}_{mi} ‖}^{2} + \frac{{λ}_{D}}{M \times {N}_{m}} {\sum }_{m}^{M} {\sum }_{i}^{{N}_{m}} ( 1 - \frac{{ℱ}_{mi}^{L} \cdot {F}_{mi}}{‖ {ℱ}_{mi}^{L} ‖ ‖ {F}_{mi} ‖} ) \qquad ( 24 )$$

The first two terms correspond to the energy and the forces on the basis of mean squared deviations of predicted values with reference data. The last term penalizes a deviation of the direction of the latent force vectors with ground-truth force vectors. Here, a cosine similarity loss function may be leveraged to minimize (1−cos(α))∈[0,2], where a is an angle between Fᵢ^{L} and Fᵢ for each atom i of a snapshot m of an MD trajectory. The λ_{E}, λ_{F}, and λ_{D} are hyperparameters that determine contributions of energy, force, and latent force direction losses, respectively, in the (total) loss function [symbol] .

A mini-batch gradient descent algorithm (with Adam optimizer) may be used to minimize the loss function with respect to trainable parameters. The trainable parameters may be built in the learned functions noted with a “ϕ” symbol. A fully connected neural network with SiLU nonlinearity for all ϕ functions may be used throughout the message passing layer. One exception may include ϕ_{rbf}, which is a single linear layer. Bias parameters may be avoided in ϕ_{f} and ϕᵣ′ in order to propagate the radial cutoff throughout the geometric MPNN 194. In some examples, such as when using data from the ANI model, a normalization layer on the atomic features may be employed at every message passing layer to facilitate training stability. The embodiments herein may use L=3 message passing layers, nf=128 features, and nb=20 basis sets, which is comparable to other models. However, it will be appreciated that greater or fewer message passing layers, features, and/or basis sets may be selected within the scope of the present disclosure. Other hyperparameters may be selected to be tailored to a given system type (see Table 1 for examples discussed hereinbelow). For example, in training SchNet for the below discussed example utilizing hydrogen combustion reaction data, nf=128 features everywhere across five interaction layers. Other hyperparameter selections for the below discussed examples are the same as utilized for the geometric MPNN, excepting a force coefficient in the loss function, where a lower λ_{F}=10 may perform better than larger coefficients (e.g., λ_{F}=20, 30, or 50; see Table 1).

TABLE 1
Hyperparameters utilized for example datasets discussed herein.

| | | | | | Learning | Cutoff |
|---|---|---|---|---|---|---|
| | | | | Learning | rate | radius (r_{c}, |
| Dataset | λ_{E} | λ_{F} | λ_{D} | rate | decay | in Å) |
| MD17 | 1 | 50 | 1 | 1 × 10⁻³ | 0.7 | 5 |
| MD17/CCSD(T) | 1 | 50 | 1 | 1 × 10⁻³ | 0.7 | 5 |
| ANI | 1 | 0 | 0 | 1 × 10⁻⁴ | 0.7 | 5 |
| Methane | 1 | 30 | 1 | 1 × 10⁻³ | 0.7 | 5 |
| combustion | | | | | | |
| reaction | | | | | | |
| Hydrogen | 1 | 20 | 1 | 5 × 10⁻⁴ | 0.7 | 5 |
| combustion | | | | | | |
| reaction | | | | | | |

Hereinbelow, performance of an exemplary embodiment of the geometric MPNN provided herein on various relatively small molecules is evaluated. In one example, the performance of the geometric MPNN is evaluated on data generated from MD trajectories using density functional theory (DFT) for nine small organic molecules from the MD17 benchmark. Despite reported outliers in calculated energies associated with this data, the original version of MD17 is employed to predict energy and forces for each “dedicated” molecule separately. To train the geometric MPNN, a data size of 950 for training and 50 for validation is selected, with remaining data utilized for test. This data split uses less training samples than that used by kernel methods such as sGDML and FCHL19, and is supported by other emerging ML models that utilize equivariant operators and train on a (relatively) low number of samples (e.g., NequIP and PaiNN).

Table 2 shows the performance of the geometric MPNN for both energy and forces on the test set, indicating outperformance of other invariant deep learning models (e.g., SchNet, PhysNet, and DimeNet) and even in some cases state-of-the-art equivariant models such as NequIP and PaiNN. Furthermore, the geometric MPNN may remain computationally efficient and scalable relative to methods which incorporate higher order tensors in equivariant operators and/or are trained on a revised version of MD17 dataset, while still retaining chemical accuracy (<0.5 kcal/mol).

TABLE 2
Performance of various models (Models 1-8 label SchNet, PhysNet,
DimeNet, FCHL19, sGDML, NequIP, PaiNN, and the geometric MPNN, respectively) in terms of
mean absolute error (MAE) for prediction of energies (E, in kcal/mol) and forces (F, in kcal/mol/Å)
of molecules in the MD17 dataset. Results include standard deviations, which are defined by
averaging over four random splits of the data. Best results within the standard deviation range of
the geometric MPNN are indicated in bold.

| | | Model | Model | Model | Model | Model | Model | Model | Model |
|---|---|---|---|---|---|---|---|---|---|
| System | | 1 | 2 | 3 | 4 | 5 | 6 | 7 | 8 |
| Aspirin | E | 0.370 | 0.230 | 0.204 | 0.182 | 0.19 | — | 0.159 | 0.168 ± |
| | | | | | | | | | 0.019 |
| | F | 1.35 | 0.605 | 0.499 | 0.478 | 0.68 | 0.348 | 0.371 | 0.348 ± |
| | | | | | | | | | 0.014 |
| Ethanol | E | 0.08 | 0.059 | 0.064 | 0.054 | 0.07 | — | 0.063 | 0.061 ± |
| | | | | | | | | | 0.009 |
| | F | 0.39 | 0.160 | 0.230 | 0.136 | 0.33 | 0.208 | 0.230 | 0.211 ± |
| | | | | | | | | | 0.036 |
| Malonaldehyde | E | 0.13 | 0.094 | 0.104 | 0.081 | 0.10 | — | 0.091 | 0.096 ± |
| | | | | | | | | | 0.013 |
| | F | 0.66 | 0.319 | 0.383 | 0.245 | 0.41 | 0.337 | 0.319 | 0.323 ± |
| | | | | | | | | | 0.019 |
| Naphthalene | E | 0.16 | 0.142 | 0.122 | 0.117 | 0.12 | — | 0.117 | 0.118 ± |
| | | | | | | | | | 0.002 |
| | F | 0.58 | 0.310 | 0.215 | 0.151 | 0.11 | 0.096 | 0.083 | 0.084 ± |
| | | | | | | | | | 0.006 |
| Salicylic acid | E | 0.20 | 0.126 | 0.134 | 0.114 | 0.12 | — | 0.114 | 0.115 ± |
| | | | | | | | | | 0.008 |
| | F | 0.85 | 0.337 | 0.374 | 0.221 | 0.28 | 0.238 | 0.209 | 0.197 ± |
| | | | | | | | | | 0.004 |
| Toluene | E | 0.12 | 0.100 | 0.102 | 0.098 | 0.10 | — | 0.097 | 0.094 ± |
| | | | | | | | | | 0.005 |
| | F | 0.57 | 0.191 | 0.216 | 0.203 | 0.14 | 0.101 | 0.102 | 0.088 ± |
| | | | | | | | | | 0.002 |
| Uracil | E | 0.14 | 0.108 | 0.115 | 0.104 | 0.11 | — | 0.104 | 0.107 ± |
| | | | | | | | | | 0.004 |
| | F | 0.56 | 0.218 | 0.301 | 0.105 | 0.24 | 0.172 | 0.140 | 0.149 ± |
| | | | | | | | | | 0.003 |
| Azobenzene | E | — | 0.197 | — | — | 0.092 | — | — | 0.142 ± |
| | | | | | | | | | 0.003 |
| | F | — | 0.462 | — | — | 0.409 | — | — | 0.138 ± |
| | | | | | | | | | 0.010 |
| Paracetamol | E | — | 0.181 | — | — | 0.153 | — | — | 0.135 ± |
| | | | | | | | | | 0.004 |
| | F | — | 0.519 | — | — | 0.491 | — | — | 0.263 ± |
| | | | | | | | | | 0.010 |

In another example, the geometric MPNN is trained on coupled-cluster theory with single and double excitations (CCSD) and coupled-cluster theory with single, double, and perturbative triple excitations (CCSD(T)) data reported for five small molecules (CCSD/CCSD(T) being the so-called “gold standard” of theory). In an exemplary embodiment, the geometric MPNN may generate machine learned potential energy surfaces therefrom at high reference accuracy with a computationally affordable number of (training) samples. In the below benchmark data, training and test data splits are fixed at 1000 training data and 500 test data, as provided by the authors of the MD17 data.

In Table 3, results from the geometric MPNN are compared with NequIP and sGDML. As shown, the geometric MPNN not only outperforms the best reported prediction performance for three of the five molecules, but also remains competitive within a range of uncertainties for the remaining two molecules, and is robustly improved compared to kernel methods.

TABLE 3
Performance of various models (Models 5, 6, and 8 label NequIP,
sGDML, and the geometric MPNN, respectively) in terms of MAE
for the prediction of energies (E, in kcal/mol) and forces
(F, in kcal/mol/Å) of molecules at CCSD or CCSD(T) accuracy.
50 snapshots (samples) of the training data are randomly selected
as a validation set, and a performance of the geometric MPNN
is averaged over four random splits to define standard deviations.
Best results within the standard deviation range of the geometric
MPNN are indicated in bold.

| System | | Model 6 | Model 5 | Model 8 |
|---|---|---|---|---|
| Aspirin | E | 0.158 | — | 0.100 ± 0.007 |
| | F | 0.761 | 0.339 | 0.356 ± 0.019 |
| Benzene | E | 0.003 | — | 0.004 ± 0.001 |
| | F | 0.039 | 0.018 | 0.011 ± 0.001 |
| Ethanol | E | 0.050 | — | 0.037 ± 0.019 |
| | F | 0.350 | 0.217 | 0.236 ± 0.030 |
| Malonaldehyde | E | 0.248 | — | 0.045 ± 0.004 |
| | F | 0.369 | 0.369 | 0.285 ± 0.038 |
| Toluene | E | 0.030 | — | 0.014 ± 0.001 |
| | F | 0.210 | 0.101 | 0.080 ± 0.005 |

Hereinbelow, performance of an exemplary embodiment of the geometric MPNN provided herein is evaluated on various relatively small molecules with relatively large chemical variations. In one example, the geometric MPNN is trained using the ANI-1 dataset to predict energies for a relatively large and diverse set of 20 million conformations sampled from ˜58000 small molecules with up to eight heavy atoms. The ANI-1 dataset is considered challenging to capture at desirably high accuracy at least because: (i) molecular compositions and conformations therein are highly diverse, with a total number of atoms in each ranging from 2 to 26, and with total energies spanning a range of near 3×10⁵ kcal/mol; and (ii) only energy information is provided, so a well-trained network desirably extracts information more efficiently from the dataset to outcompete other, more data-intensive invariant models. As exemplified in Table 4, the geometric MPNN performs well on a diverse dataset and may thereby be more transferable to unseen data and concomitantly wider application domains.

TABLE 4
Performance of the geometric MPNN on relatively small fractions
of the 20-million molecule ANI-1 dataset (which includes molecules
with a range of sizes and conformations) as compared with artificial
neural networks (ANNs). Energies are presented in terms of MAE
for the prediction of the energies (in kcal/mol/atom).

| | Model | | |
|---|---|---|---|
| | ANI-1 | Geometric | Geometric |
| | (ANNs) | MPNN | MPNN |
| Training set size (in | 20000000 | 2000000 | 1000000 |
| number of molecules) | | | |
| Energies | 1.30 | 0.65 | 0.85 |

By utilizing only 10% of the data (2000000 samples of the full ANI-1 dataset), the geometric MPNN yields an MAE in energies of 0.65 kcal/mol, near standard definitions of chemical accuracy (e.g., <0.5 kcal/mol) and halving the MAE compared to ANNs using the full ANI-1 dataset. Even with only 5% of the data (1000000 samples of the full ANI-1 dataset), the geometric MPNN yields an MAE in energies of 0.85 kcal/mol, which still exceeds the performance of the ANNs trained with the full ANI-1 dataset. However, unlike other data experiments, atomic forces are not reported with the ANI-1 dataset. Although the geometric MPNN may be trained without taking advantage of additional information for the atomic environments and the force and direction components of the loss function that are also not operative, the directional information may be considered a significant component of the atomic feature representation regardless of a tensor order of output properties.

Hereinbelow, performance of an exemplary embodiment of the geometric MPNN provided herein is evaluated on methane combustion reaction data. The methane combustion reaction data constitutes a more challenging task for the ML models due to the complex nature of reactive species which are often high in energy, transient, and far from equilibrium (e.g., free radical intermediates). Such “stress tests” are important for driving ab initio MD (AIMD) simulations in which even relatively low-run DFT functionals are time-consuming and limited to small system sizes. The dataset provided by Zeng et al. (Zeng, J.; Cao, L.; Xu, M.; Zhu, T.; and Zhang, J. Z H, Nature Comm., 11(1):1-9, 2020) is utilized, which is generated through an active learning procedure and follows the same split of 13315 snapshots to define the test set. Performance of the geometric MPNN on 100%, 10%, and 1% of the remaining data is evaluated for training and validation. Table 5 shows that when the geometric MPNN is trained on all available data, errors in energies and forces may be driven down significantly. As a result, an MAE in the energies of 0.50 kcal/mol/atom in energies and an MAE in the forces of 1.20 kcal/mol/Å may be obtained, thereby decreasing error in energy and force predictions by 85% and 57%, respectively, compared to the model in Zeng et al. Utilizing 10% of the data, the geometric MPNN has an MAE which remains close to chemical accuracy, and even using only 1% of the data, the geometric MPNN maintains superiority in performance as compared to the DeepMD model originally trained with the full dataset.

TABLE 5
Performance of the geometric MPNN as compared with DeepMD on
13315 test configurations of the methane combustion reaction
in terms of MAE for the prediction of energies (in kcal/mol/atom)
and forces (in kcal/mol/Å). Specifically, an amount of
training data is systematically and sequentially reduced by
two orders of magnitude using the geometric MPNN (for which
553997 energy and force values are available from the original
dataset) and compared with the original dataset of 578731
energy and force values used in Zeng et al.

| | Model | | | |
|---|---|---|---|---|
| | | Geometric | Geometric | Geometric |
| | DeepMD | MPNN | MPNN | MPNN |
| Training set size | 578731 | 553997 | 55399 | 5539 |
| (in number of | | | | |
| molecules) | | | | |
| Energies | 3.227 | 0.497 | 0.689 | 0.835 |
| Forces | 2.77 | 1.20 | 1.83 | 2.66 |

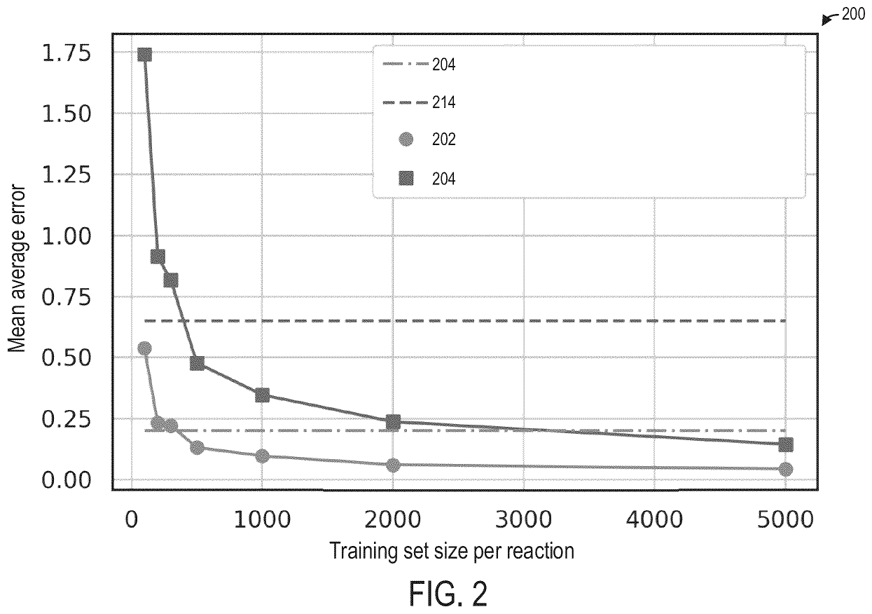

Hereinbelow, performance of an exemplary embodiment of the geometric MPNN provided herein is evaluated on the hydrogen combustion reaction. Referring now to FIG. 2, a plot 200 depicting the MAE (indicated along an ordinate) in energies and forces averaged over 16 independent reactions or “sub-reactions” (see below) as a function of a number of training samples used for each reaction (indicated along an abscissa) determined via SchNet and the geometric MPNN is shown. Specifically, the MAE in energies predicted by the geometric MPNN is indicated by a curve 202 and the MAE in forces predicted by the geometric MPNN is indicated by a curve 212. Further, the MAE in energies predicted by SchNet at a (full) training set size of 5000 sub-reactions is indicated by a dashed line 204 and the MAE in forces predicted by SchNet at the (full) training set size of 5000 sub-reactions is indicated by a dashed line 214.

The hydrogen combustion reaction data utilized to obtain the values plotted in FIG. 2 are newly generated and probe reactive pathways of hydrogen and oxygen atoms through the combustion reaction mechanism reported by Li et al. (Li, J.; Zhao, Z.; Kazakov, A.; and Dryer, F. L., Int. J. Chem. Kinet., 36(10):566-575, 2004). The generated data is analyzed with calculated intrinsic reaction coordinate (IRC) scans of 19 bimolecular sub-reactions from Bertels et al. (Bertels, L. W.; Newcomb, L. B.; Alaghemandi, M.; Green, J. R.; and Head-Gordon, M., J. Phys. Chem. A, 124(27):5631-5645, 2020). Excluding three reactions which are relatively trivial from a chemical perspective (diatomic dissociation or recombination reactions), configurations and energies and forces for reactant, transition, and product states are obtained for the remaining 16 out of the 19 bimolecular sub-reactions. The IRC scans are then augmented with normal mode displacements and AIMD simulations to sample configurations adjacent to the reaction path. All calculations are conducted at the ωB97M-V/cc-pVTZ level of theory, and the dataset includes a total of 280000 potential energies and 1240000 nuclear force vectors.

The geometric MPNN is trained on a complete reaction network of the hydrogen combustion reaction data by sampling training, validation, and test sets randomly formulated from the total data. Validation and test set sizes are fixed to 1000 data points (samples) per reaction, and a training set size varies in a range of 100 to 5000 data points (samples) per reaction. A resultant accuracy of the geometric MPNN on the test set for both energy and forces is indicated in plot 200 of FIG. 2 (comparing curve 202 to dashed line 204 and curve 212 to dashed line 214, respectively). Specifically, the geometric MPNN may outperform the (invariant) SchNet model with slightly less than one order of magnitude smaller training data (500 vs. 5000 samples per reaction), and may further be capable of achieving chemical accuracy in as little as 200 data points per reaction. In other deep learning approaches for reactive chemistry, abrupt changes in force magnitudes may give rise to multimodal distributions of data, which may introduce covariate shift in model training. In contrast, in the geometric MPNN, a better representation of atomic environments using the latent force directions may increase an amount of attention that one atom gives to immediate neighbors thereof. As a result, the performance of the geometric MPNN in prediction of forces for reactive systems may benefit most from the directional information provided by atoms that break or form new bonds.

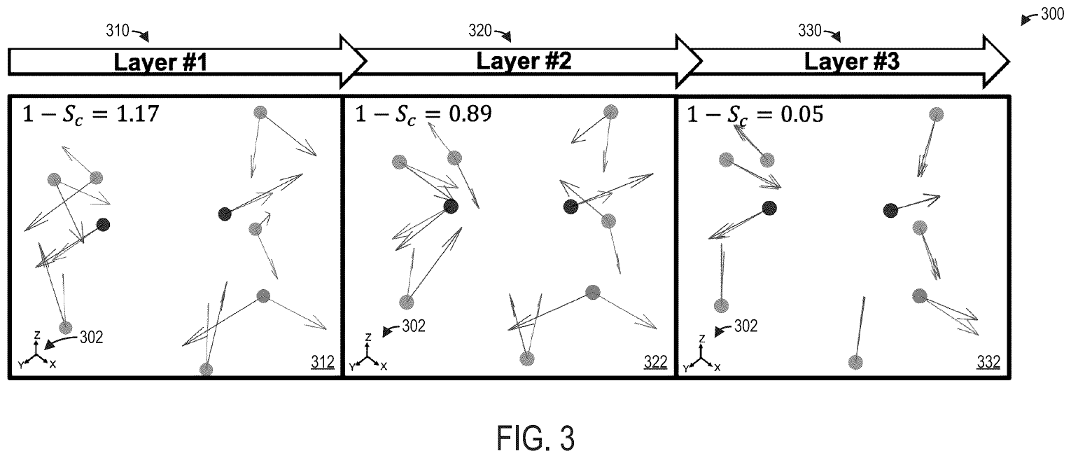

As another example, and referring now to FIG. 3, a schematic diagram 300 depicting latent force vectors (in blue) and normalized reference vectors (in green) exerted on each atom of an ethanol molecule from the MD17 dataset at consecutive message passing layers 310, 320, and 330 of the geometric MPNN is shown. Snapshots 312, 322, and 332 of the aspirin molecule following message passing at the message passing layers 310, 320, and 330, respectively, wherein an element of each atom is indicated by color (red indicates oxygen, gray indicates hydrogen, and black indicates carbon). A set of reference axes 302 indicating an x-axis, a y-axis, and a z-axis is provided for describing relative positioning of the atoms and vectors shown and for comparison between the snapshots 312, 322, and 322. Each of the snapshots 312, 322, and 322 is further tagged with 1−S_{c}∈[0, 2], indicating an average cosine distance of the latent and normalized reference force vectors for all atoms in a test set of aspirin data (determined via CCSD). From left to right (e.g., from the snapshot 312 to the snapshot 322 to the snapshot 332), cosine similarity (S_{c}) increases (that is, the cosine distance decreases) following each message passing layer (e.g., 310, 320, 330) of the geometric MPNN.

Within each of the message passing layers 310, 320, and 330, latent atomic forces may be constructed to collect directional information for each atom. As discussed above, the latent space may agree with ground-truth force vectors if guided by appropriate loss functions. As an example, the snapshots 312, 322, and 332 depict the latent force vectors of the message passing layers 310, 320, and 330, respectively, for the ethanol molecule from the test set of MD17 data. More precisely, a snapshot of the aspirin molecule is input into the geometric MPNN and 312, 322, and 332 indicate updates to the snapshot. In the (first) message passing layer 310, opposite directions and orthogonality may be observed. However, after the (last) message passing layer 330 transformation, most of the constructed latent force vectors may be in the same direction as the true reference forces, as quantified by the average cosine distance on the test set of 1.17, 0.89, and 0.05 for the message passing layers 310, 320, and 330, respectively. Precise directions of atomic forces indicate that the geometric MPNN may also adequately learn force magnitudes and signs for pairwise interactions (based on equations (9) and (10). Such accuracy indicates that describing the latent space based on Newton's second and third laws may enable the geometric MPNN to take advantage of underlying physics of interatomic interactions.

The geometric MPNN may allow for a linear scaling in computational complexity with respect to a number of atoms in a given system. To give a better sense of computational efficiency, a time utilized to train on the aspirin molecule from the MD17 dataset may be compared with the same calculation using the NequIP model. As reported by Batzner et al. (Batzner, S.; Smidt, T. E.; Sun, L.; Mailoa, J. P.; Kornbluth, M.; Molinari, N.; and Kozinsky, B., “Se(3)-equivariant graph neural networks for data-efficient and accurate interatomic potentials,” 2021), a complete training on the MD17 data to converge to a best performance of NequIP takes up to 8 days. In contrast, the geometric MPNN utilized 12 hours to give state-of-the-art performance on a GeForce® RTX™ 2080 Ti, a graphic processing unit which is 73% as fast as the Tesla V100 that is used for evaluating the NequIP model when a straightforward comparison of similar rank of contributed tensors is used by both methods. However, as higher order tensors may increase a computation time for a boost in performance, analysis from a cost-benefit perspective may be undertaken to determine a best level of ML models for accuracy versus computational resources.

Aside from training time to facilitate model development and reduce testing time, computation time per atomic environment may also be critical for applying trained models in an MD simulation. For example, a computation time for processing a snapshot of an MD trajectory of a small molecule by the geometric MPNN disclosed herein may be 4 ms (˜3 ms on a Tesla V100) for a small molecule of 20 atoms. Considering an average time of 16 ms for NequIP to process a molecule of 15 atoms, the geometric MPNN disclosed herein demonstrates a significant speedup. In addition, PaiNN may be considered closest to the geometric MPNN disclosed herein in terms of computational complexity, yet PaiNN does not encode additional physical knowledge in message passing operators. As a result, PaiNN may include about 20% more optimized parameters (600000 parameters in PaiNN vs. 500000 parameters in the geometric MPNN disclosed herein). This difference may lead to a higher computational cost with an equally efficient implementation of code. Nevertheless, the above discussed prediction times may be significantly smaller than ab initio calculations even for a snapshot of a small molecule in an MD trajectory, which may be on an order of minutes to hours.

In this way, a geometric MPNN based on Newton's equations of motion is provided which may predict an energy and forces of an MD trajectory with higher accuracy and at a more efficient timescale as compared to other deep learning models. Specifically, the disclosed geometric MPNN may achieve greater accuracy and/or competitive performance relative to other state-of-the-art invariant and equivariant models (as discussed above). A technical effect of utilizing Newton's laws of motion to design an architecture of the geometric MPNN is that excess operations may be avoided while providing a more understandable and interpretable latent space to carry out the predictions.

The geometric MPNN may take advantage of geometric message passing and a rotationally equivariant latent space, which may scale linearly with a size of the system. A technical effect of such linear scaling is that high accuracy may be achieved without significant computation or memory overhead. For example, the geometric MPNN may utilize less data and still outperform kernel methods. Given such better scalability (e.g., as compared to kernel methods), the training data may be expanded, for example, via smart sampling methods such as active learning, and a potential energy surface of a given chemical compound space may be more efficiently explored. The methane combustion reaction discussed above may be considered a proof of evidence, as the training data is a result of active learning sampling. If such sampling is initiated with predictions from the geometric MPNN, higher performance may be achieved with a lower number of queries. Moreover, high data efficiency may result in ML force field models comparable with accuracy levels of first principles methods such as CCSD(T) at a complete basis set limit (CCSD(T)/CBS) with competitive performances as state-of-the-art kernel-based methods using significantly less training data. For example, for small organic molecules, a performance of the geometric MPNN for training on CCSD(T) data may be competitive or better than other, state-of-the-art models by a significant margin (e.g., at least 10%). Inspired by other physical operations that incorporate higher order tensors, the geometric MPNN may further be extended to construct a more distinguishable latent space of many-body features. As formulated herein, a performance of the geometric MPNN on MD trajectories from combustion reactions may exhibit chemical accuracy even considering the challenges of chemical reactivity.

### Examples

In a first example, a method for a trained neural network can include: acquiring a plurality of inputs comprising a plurality of initial parameters for a polyatomic system; passing the plurality of inputs through one or more rotationally equivariant message passing layers of the trained neural network to generate a plurality of outputs comprising an update to each of the plurality of inputs; and determining a potential energy of the polyatomic system based on the plurality of outputs, wherein each of the one or more rotationally equivariant message passing layers is constructed from one or more symmetric message functions. The method can be executed by a processor executing instructions that are store by one or more tangible, non-transitory storage medium.

A second example can include the first example wherein each of the one or more symmetric message functions includes a radial Bessel function, a polynomial cutoff function, and a pair of multilayer perceptrons.

A third example can include the first example wherein the plurality of initial parameters comprises each of a plurality of atomic feature arrays, a plurality of latent force vectors, a plurality of total force vectors, a plurality of interatomic force vectors, a plurality of displacement vectors, and a plurality of interatomic distances for the polyatomic system.

A fourth example can include the third example wherein the plurality of atomic feature arrays is rotationally invariant.

In a fifth example, a system can include: a memory storing a trained geometric message passing neural network configured to predict potential energies and forces of atomic systems, the trained geometric message passing neural network comprising one or more rotationally equivariant message passing layers constructed from one or more symmetric message functions; and a processor configured with instructions in non-transitory memory that when executed cause the processor to: receive a plurality of initial parameters of an atomic system of interest; and pass the plurality of initial parameters through the one or more rotationally equivariant message passing layers to predict each of a potential energy and a plurality of forces of the atomic system of interest.

A sixth example can include the fifth example wherein predicting each of the potential energy and the plurality of forces of the atomic system of interest comprises: generating a plurality of latent force vectors based on Newton's third law; and minimizing an average cosine distance between the plurality of latent force vectors and a plurality of ground-truth force vectors of the atomic system of interest.

In a seventh example, a method for a neural network can include: training the neural network to predict each of a potential energy and a plurality of forces of an atomic system by: acquiring a set of training data comprising a plurality of samples of the atomic system; passing the set of training data through one or more rotationally equivariant message passing layers constructed from one or more symmetric message functions; and penalizing deviations of the potential energy and the plurality of forces by minimizing a loss function with respect to a plurality of trainable parameters; receiving a plurality of initial parameters of the atomic system; and predicting each of the potential energy and the plurality of forces of the atomic system by updating the plurality of initial parameters with the trained neural network. The method can be executed by a processor executing instructions that are store by one or more tangible, non-transitory storage medium.

An eighth example can include the seventh example wherein minimizing the loss function with respect to the plurality of trainable parameters comprises minimizing an average cosine distance between a plurality of latent force vectors of the atomic system and a plurality of normalized reference force vectors of the atomic system.

A ninth example can include the seventh example wherein a size of the set of training data is 1-10% of a size of a set of training data used to train other message passing neural networks and achieve comparable accuracy.

A tenth example can include the ninth example wherein the trained neural network predicts the potential energy and the plurality of forces faster than the other message passing neural networks for a given computing device implementing the trained neural network.

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers.

One or more aspects of the disclosure may be embodied in computer-usable data and computer-executable instructions, such as in one or more program modules, executed by one or more computers (including monitoring modules), or other devices. Generally, program modules include routines, programs, objects, components, data structures, etc. that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGAs, and the like.

Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

The previously described versions of the disclosed subject matter have many advantages that were either described or would be apparent to a person of ordinary skill. Even so, these advantages or features are not required in all versions of the disclosed apparatus, systems, or methods.

Additionally, this written description makes reference to particular features. It is to be understood that the disclosure in this specification includes all possible combinations of those particular features. Where a particular feature is disclosed in the context of a particular aspect or example, that feature can also be used, to the extent possible, in the context of other aspects and examples.

Also, when reference is made in this application to a method having two or more defined steps or operations, the defined steps or operations can be carried out in any order or simultaneously, unless the context excludes those possibilities.

Although specific examples of the invention have been illustrated and described for purposes of illustration, it will be understood that various modifications may be made without departing from the spirit and scope of the invention. Accordingly, the invention should not be limited except as by the appended claims.

1. A method for a trained neural network, the method comprising:
acquiring a plurality of inputs comprising a plurality of initial parameters for a polyatomic system;
passing the plurality of inputs through one or more rotationally equivariant message passing layers of the trained neural network to generate a plurality of outputs comprising an update to each of the plurality of inputs; and
determining a potential energy of the polyatomic system based on the plurality of outputs,
wherein each of the one or more rotationally equivariant message passing layers is constructed from one or more symmetric message functions.

2. The method of claim 1, wherein each of the one or more symmetric message functions includes a radial Bessel function, a polynomial cutoff function, and a pair of multilayer perceptrons.

3. The method of claim 1, wherein the plurality of initial parameters comprises each of a plurality of atomic feature arrays, a plurality of latent force vectors, a plurality of total force vectors, a plurality of interatomic force vectors, a plurality of displacement vectors, and a plurality of interatomic distances for the polyatomic system.

4. The method of claim 3, wherein the plurality of atomic feature arrays is rotationally invariant.

5. One or more tangible, non-transitory storage medium storing executable instructions that, when executed by a processor, cause the processor to perform the method of claim 1.

6. A system, comprising:
a memory storing a trained geometric message passing neural network configured to predict potential energies and forces of atomic systems, the trained geometric message passing neural network comprising one or more rotationally equivariant message passing layers constructed from one or more symmetric message functions; and
a processor configured with instructions in non-transitory memory that when executed cause the processor to:
receive a plurality of initial parameters of an atomic system of interest; and
pass the plurality of initial parameters through the one or more rotationally equivariant message passing layers to predict each of a potential energy and a plurality of forces of the atomic system of interest.

7. The system of claim 6, wherein predicting each of the potential energy and the plurality of forces of the atomic system of interest comprises:
generating a plurality of latent force vectors based on Newton's third law; and
minimizing an average cosine distance between the plurality of latent force vectors and a plurality of ground-truth force vectors of the atomic system of interest.

8. A method for a neural network, the method comprising:
training the neural network to predict each of a potential energy and a plurality of forces of an atomic system by:
acquiring a set of training data comprising a plurality of samples of the atomic system;
passing the set of training data through one or more rotationally equivariant message passing layers constructed from one or more symmetric message functions; and
penalizing deviations of the potential energy and the plurality of forces by minimizing a loss function with respect to a plurality of trainable parameters;
receiving a plurality of initial parameters of the atomic system; and
predicting each of the potential energy and the plurality of forces of the atomic system by updating the plurality of initial parameters with the trained neural network.

9. The method of claim 8, wherein minimizing the loss function with respect to the plurality of trainable parameters comprises minimizing an average cosine distance between a plurality of latent force vectors of the atomic system and a plurality of normalized reference force vectors of the atomic system.

10. The method of claim 8, wherein a size of the set of training data is 1-10% of a size of a set of training data used to train other message passing neural networks and achieve comparable accuracy.

11. The method of claim 10, wherein the trained neural network predicts the potential energy and the plurality of forces faster than the other message passing neural networks for a given computing device implementing the trained neural network.

12. One or more tangible, non-transitory storage medium storing executable instructions that, when executed by a processor, cause the processor to perform the method of claim 8.
